(19) United States
(12) Patent Application Publication
(10) Pub. No.: US 2004/0260320 A1
(43) Pub. Date: Dec. 23, 2004

(54) Disposable separator for separating the epithelium layer from the cornea of an eye

(76) Inventors: James R. Lisk JR., Suwanee, GA (US); Ming-Kok Tai, Lawrenceville, GA (US); Scott M. Hampton, Cumming, GA (US)

Correspondence Address: GARDNER GROFF, P.C., PAPER MILL VILLAGE, BUILDING 23, 600 VILLAGE TRACE, SUITE 300, MARIETTA, GA, 30067, US

(21) Appl. No.: 10/732,890

(22) Filed: Dec. 10, 2003

Related U.S. Application Data
(60) Provisional application No. 60/432,305, filed on Dec. 10, 2002.

(51) Int. Cl. A61F 9/00

(52) U.S. Cl. 606/166

(57) ABSTRACT

A separator constructed of a polymeric material is used with a surgical device that separates the epithelium of a cornea from the underlying Bowman's layer of an eye of a patient. The surgical device includes a positioning ring for temporary attachment to the eye and is structured to present and expose the cornea to be separated. The separator support is structured and disposed to carry the separator. A drive is operably connected to the separator support for causing movement of the separator across the positioning ring and for causing oscillating movement of said separator.

### CROSS-REFERENCE TO RELATED APPLICATION

This application claims the benefit of U.S. Provisional Patent Application Ser. No. 60/432,305, filed Dec. 10, 2002, which application is hereby incorporated herein by reference in its entirety.

### FIELD OF THE INVENTION

This invention relates to a device for optical surgery, and in particular to a disposable separator for separating the epithelium layer of a cornea from the underlying Bowman's layer.

### BACKGROUND OF THE INVENTION

Microkeratome blades are widely used in LASIK (Laser-Assisted In Situ Keratomilousis) procedures. LASIK permanently changes the shape of the cornea, the clear covering of the front of the eye, using an excimer laser. The microkeratome is used to cut a corneal flap containing the epithelium, Bowman's layer, and a portion of the stroma by slicing through the stroma, dividing it into at least two distinct portions. A hinge of uncut corneal tissue is typically left at one end of this flap. The flap is folded back revealing the penetrated stroma, the middle section of the cornea. Pulses from a computer-controlled laser vaporize a portion of the stroma and the flap is replaced. It is important that the blade used during the LASIK procedure is sharp, otherwise the quality of the procedure and the healing time are poor. Additionally, the blade has to be exceedingly sharp in order to produce consistent and reproducible flaps.

Known microkeratome blades are typically formed of either stainless or low-carbon steel. A variety of other materials, including diamond, sapphire, tungsten, ceramic, and silicon carbide, have been proposed for use in microkeratome blades. Among the known materials, diamond is believed to have the best cutting capacity due to its great hardness, because the cutting edge can be sharpened to a very small radius of curvature, for example in the nanometer range. Disadvantages are, however, the high material cost and the difficulties in applying the diamond as a cutting edge on a blade.

A blade made of stainless steel, on the other hand, can be manufactured in a comparatively simple way, and offers considerable cost advantages. However, while stainless steel blades are cheaper to manufacture than diamond blades, they are not so inexpensive as to render them “disposable” in all instances. Stainless steel blades are, therefore, sometimes autoclaved after a use and reused on another patient. While autoclaving is generally considered an effective method of sterilization, it is not foolproof, and only one-time use of blades can ensure that each blade is entirely free of infection or physical defects.

Because the “sharpness” of the blade, until now, has been considered to be the most important characteristic of the blade for achieving a precise and consistent corneal resection, materials cheaper than stainless steel, such as plastics, have not been considered for use in microkeratome blades, as these materials are typically too soft to achieve the required edge sharpness. Instead, the art has focused on various methods of manufacturing ever-more sharper steel blades, resulting in more complex and expensive manufacturing processes, and rendering re-use of blades more tempting economically for practitioners. For instance, it has been proposed to melt the cutting edge of a metallic blade body by laser beam treatment and to rapidly cool it off in a water bath. In this way, the cutting edge is amorphized and can then be sharpened to a radius of curvature less than several ten nanometers. It has also been proposed to produce sharper blades by providing a blade having a carrier portion and a thin-walled cover portion made of amorphous metal, which is joined to the carrier portion and forms a cutting edge. To prevent multiple uses of a blade, it has been proposed to magnetically encode the blade upon its first use, and to provide a microkeratome machine that will not accept a blade if a subsequent use is attempted. However, such proposed equipment is complex and expensive.

Thus, there is a need in the art for a device that effects separation of the corneal epithelium, and that can be manufactured in a simple fashion from inexpensive polymeric raw materials. Additionally, it is advantageous for the blade to be configured for one time use by virtue of its material composition. It is to the provision of a device meeting these and other needs that the present invention is primarily directed.

### SUMMARY OF THE INVENTION

The present invention provides a disposable separator or blade for separating the epithelium of a cornea from the underlying Bowman's layer, the device comprising a separator fabricated from a polymeric material. The separator comprises a front portion that includes a separating edge, a rear trailing portion having a rear edge, and a pair of side edges that extend from the front and rear portions. The separating edge is sharp enough to separate the epithelium layer from Bowman's layer, but not sharp enough to cut into Bowman's layer when in contact therewith. The blade may include a blade holder that is preferably, but not necessarily, a polymeric material.

In another aspect, the invention provides a separator to be used with a handpiece drive tool or other surgical device that separates the epithelium of a cornea from the underlying Bowman's layer of an eye of a patient, the surgical device preferably including a positioning ring for temporary attachment to the eye and structured to present and expose the cornea to be separated, a separator head assembly structured and disposed to releasably engage and carry said separator, and a drive operably connected to the separator head assembly for causing movement of the separator across the positioning ring and for causing oscillating movement of said separator, said separator comprising a separating edge, said separator having a polymeric separating edge. The separator preferably includes one or more coupling features for engagement with cooperating coupling features of the drive tool. Example drive tools and methods suitable for use in connection with the separator of the present invention are disclosed in U.S. Provisional Patent Application Ser. No. 60/435,009, filed Dec. 19, 2002, and U.S. Provisional Patent Application Ser. No. 60/500,874, filed Sep. 5, 2003, which applications are hereby incorporated herein by reference.

In a preferred aspect of the invention, the polymeric material of the separator is transparent. A transparent separator will not obstruct the visual field when observing the progress of the separator through the cornea. More preferably, the polymeric material comprises a slight tint so that it is visibly different in perceived color than the epithelium.

In another preferred aspect, the separator is constructed of a polymeric material that will undergo dimensional changes if exposed to temperatures exceeding about 100° C. This can be accomplished, for example, with a polymeric material that has a Vicat softening point below about 100° C. This prevents the blade from being used after either autoclaving or steam sterilization, thus ensuring that a new, pristine and sterile blade is used on each patient. In this manner, the quality and safety of the separator can be guaranteed.

In yet another aspect of the present invention a method is provided for separating at least a portion of an epithelium from a cornea of an eye, so that an intact Bowman's layer of the cornea is exposed. The method comprises the steps (a) fixing a positioning ring to an eye so that the cornea at least partially extends therethrough; (b) moving a separator having a polymeric separating edge along a travel path that intersects at least a portion of the cornea so as to separate the epithelium from the cornea, leaving Bowman's layer intact; and (c) retracting the separator out of contact with the cornea. The edge of the separator is preferably sufficiently blunt so as not to sever Bowman's layer, but rather to separate and peel back the corneal epithelium from Bowman's layer.

In example embodiments, the present invention provides a separator that is able to separate the epithelium of a cornea from the underlying Bowman's membrane in such a way that the epithelium can be easily and precisely aligned back into its original position following the reshaping of the cornea. Example embodiments of the separator of the present invention can be manufactured cheaply and easily, such that the separator is economically disposable, thus reducing the incentive to reuse the device and create a chance of infection due to inadequate sterilization. Example embodiments of the separator of the present invention are incapable of being sterilized by autoclaving or steam sterilization after use. Optionally, however, the separator is capable of being sterilized by other means, such as, for example, exposure to electromagnetic radiation, or to chemical agents. Example embodiments of the separator of the present invention do not obstruct the visual field of the surgeon as the separator progresses through the cornea.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWING FIGURES

FIG. 1 is a perspective view of a separator according to one embodiment of the present invention.

FIG. 2 is a cross-sectional view of the first three layers of tissue of the cornea of an eye.

FIG. 3 is a partial side view of a flat leading edge portion of a separator according to an embodiment of the present invention.

FIG. 4 is a partial side view of a rounded leading edge of a separator according to another embodiment of the present invention.

FIG. 5 is a partial side view of an angled leading edge of a separator according to yet another embodiment of the present invention.

FIGS. 6A-6C are cross-sectional views of separators according to different embodiments of the present invention.

FIG. 7 is a side view of a separator assembly according to an embodiment of the present invention.

FIG. 8 is a side view of a hand piece useful in practicing the present invention.

FIG. 9 is a side view of the separator assembly in a first position slidably engaged with a hand piece secured to the eye by vacuum.

FIG. 10 is a side view of the separator assembly of FIG. 9 in a second position.

FIG. 11 is a side view of the separator assembly of FIG. 9 in a third position.

FIG. 12 is a top view of portions of the hand piece and separator assembly of FIG. 9 after the epithelium has been separated from the eye.

FIG. 13 is a cross-sectional side view of a portion of the separator assembly showing the spatial relationship between the separating edge and the applanator.

FIGS. 14A-14C show various possible configurations of the separated epithelium as the separating edge engages the cornea and causes separation of the epithelium from the Bowman's layer.

### DETAILED DESCRIPTION

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

The disclosed epithelium separator is especially suited for use in excimer laser reshaping of the cornea. It is safer than standard microkeratomes used in eye surgery, and is inexpensive enough to be a disposable, single use device, which eliminates the need for sterilization between procedures, and thus reduces the possibility of infection.

The disclosed separator is ideally suited to the unique requirements for separating the epithelium layer from the underlying Bowman's layer. While microkeratomes developed to sever the stroma for laser in situ keratomileusis were required to be extremely hard and sharp to maintain a radius of curvature as low as 1 micron at the edge, the present separator is not intended for applications requiring severing of Bowman's layer or the stroma, and therefore has no such stringent sharpness requirements and can be constructed of cheaper, softer materials. In fact, in example embodiments, the edge of the separator is sufficiently “blunt” so as not to be capable of severing Bowman's layer under normal operating conditions, but instead, only has sharpness sufficient to cleave the boundary between the epithelium and Bowman's layer. For example, the “blunt” leading edge of the separator of the present invention preferably has a radius of curvature of at least about 5 microns, and no more than about 100 microns. In further preferred embodiments, the radius of curvature of the separator edge is between about 10 microns and about 30 microns, and most preferably is between about 15 microns to about 25 microns.

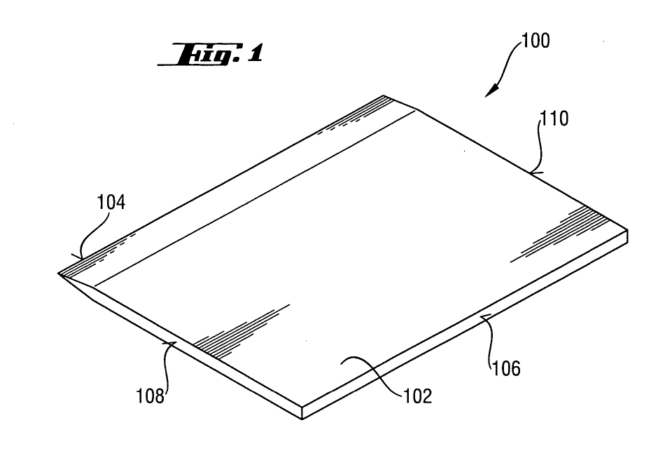

Referring now to FIG. 1, the separator 100 comprises a separator body 102 having a leading separating edge 104, a rear edge 106, and first and second side edges 108, 110 that extend from the separating edge 104 to the rear edge 106, thereby defining the body 102. In a preferred embodiment, the rear edge 106 is generally parallel to the separating edge 104. The separating edge 104 is the first portion of the separator 100 to come into contact with the cornea and effects the separation of the epithelium therefrom. At least the separating edge 104 of the separator 100 is formed of a plastic or polymeric material. In example embodiments, the entire body 102 is formed of a plastic or polymeric material.

While the dimensions and configuration of the separator are largely determined by the instrument in which they are to be used, the separator 100 is preferably less than 1000 microns in thickness. However, because the separating edge 104 must not be sharp enough to cut into Bowman's layer under normal operating conditions, it should not be so thin that excision of Bowman's layer would occur. The thickness of the separating edge 104 is preferably greater than about 200 microns, sufficient to prevent cutting into Bowman's layer under normal operating conditions.

While the separator 100 can be flat, having a rear edge 106 substantially the same width as the separating edge 104, more preferably, the rear edge 106 is thicker in dimension than the separating edge 104, the body 102 being tapered toward the separating edge. In some cases, the rear edge 106 may be an order of magnitude thicker than the separating edge 104, and even up to two orders of magnitude thicker. Such dimensions may make it easier for the surgeon to handle the separator prior to insertion into the surgical device and also aid in its stability once installed.

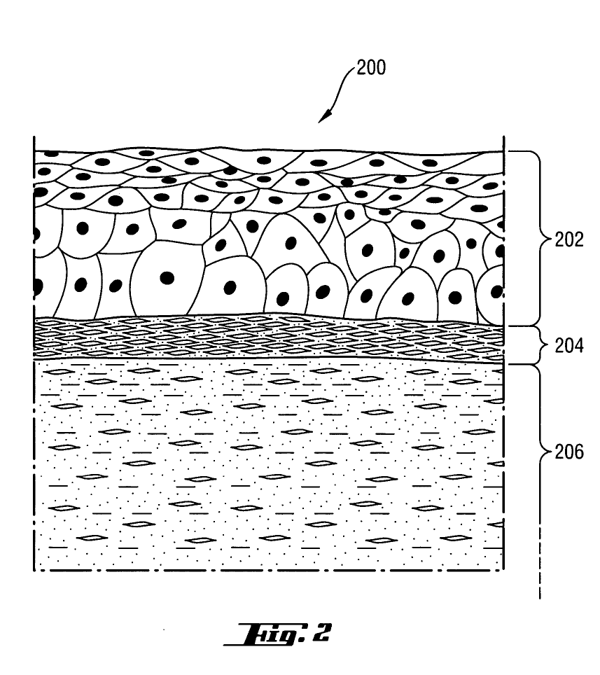

The cornea 200 of the human eye includes five layers, the outer three of which are illustrated in FIG. 2. The outer-most layer is known as the epithelium layer 202 and is typically 50 to 90 microns thick. The epithelial layer 202 is stratified, possessing 5 to 6 layers of epithelial cells, which are held together by desmosomes (not shown). Bowman's layer 204 separates the epithelium from the stroma layer 206. Bowman's membrane 204 is typically about 12 microns thick, while the stroma 206 is from 400 to 450 microns thick and makes up most of the thickness of the cornea. While the example embodiments of the present invention disclosed herein are considered optimal for use upon a human eye, it is to be understood that such a separator is easily modified for use on similar animal eyes, including eyes of most mammals and many vertebrates, such as horses, dogs, cats, elephants, sheep, and swine.

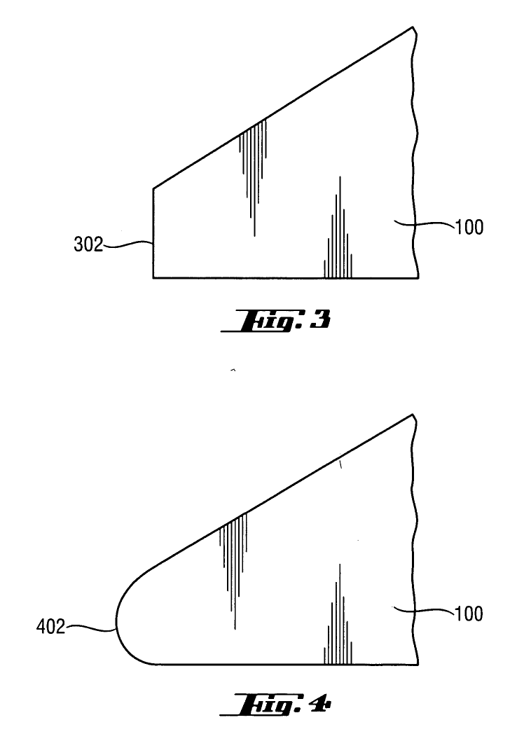

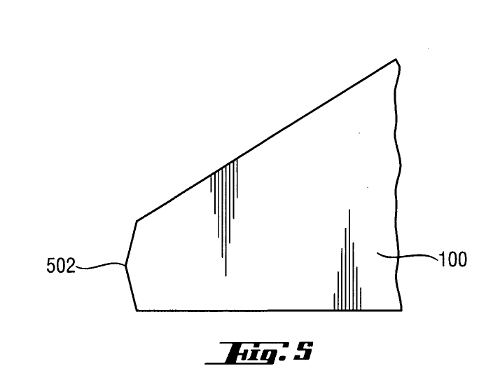

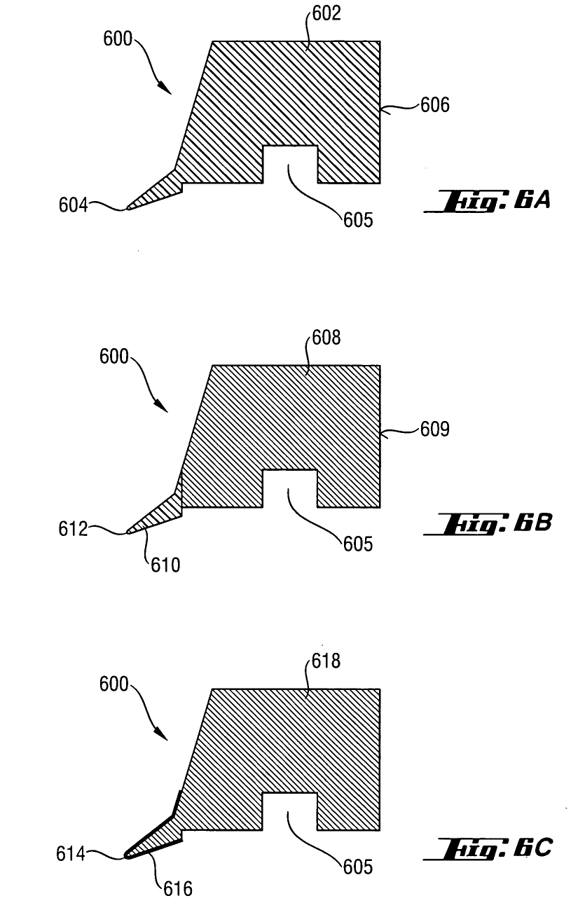

FIG. 3 shows a side view of a flat separating edge 302 portion of a separator 100 according to one embodiment of the invention. The polymeric separating edge 302 of the separator 100 should not be too thick, such that it will reduce the consistency with which the epithelial layer 202 is penetrated. The separating edge 302 preferably is about 5 to 25 micrometers thick, and more preferably about 15 micrometers thick. FIG. 4 shows a side view of a rounded separating edge 402 according to another embodiment of the separator 100. As shown in FIG. 5, the separating edge can also come to an angled point 502, provided, however, that it is not sufficiently sharp to sever Bowman's layer when used as intended.

As shown in FIG. 6A, the separator 600 need not be the flat rectangular shape shown in FIG. 1. In alternate embodiments, the separator 600 comprises a separator body 602 having a polymeric separating edge 604, a rear edge 606, and a pair of side edges (not shown) that extend from the polymeric separating edge 604 to the rear edge 606 defining the body. A notch, projection, or other surface feature 605 is preferably provided on the underside of the separator 600 or elsewhere for coupling with a cooperating support member of a surgical device for stability.

While in some embodiments the separator 600 comprises a solid body of polymeric material and optionally includes a reinforcing material therein, in alternate embodiments the separator 600 is fabricated as a polymeric coated metallic or ceramic body. For example, a metallic core 618 can be employed as a base structure upon which a polymeric or polymeric-composite material 616 is disposed. While FIG. 6C, shows a polymeric coating 616 over only the separating edge, the coating 616 may alternatively cover the entire metallic core 618. In this manner, the metal core will provide rigidity to the separator 600 whereas the polymeric material 616 will provide the blunt separating edge 614 for interaction with the cornea without the risk of severing Bowman's layer.

FIG. 6B shows another alternate embodiment of the present invention in which the separator 600 comprises a polymeric front portion 610 that includes a separating edge 612, and a metallic rear portion 608 comprising a rear edge 609. The front portion 610 is joined to the rear portion 608 in any one of a variety of known ways, such as by adhesive, thermal or solvent welding, interengaging surface features, one or more fasteners, or the like, for example. As in the embodiment shown in FIG. 6C, the metal portion 608 will provide rigidity to the separator 600 whereas the polymeric portion 610 will provide the blunt separating edge 612 for contact with the cornea.

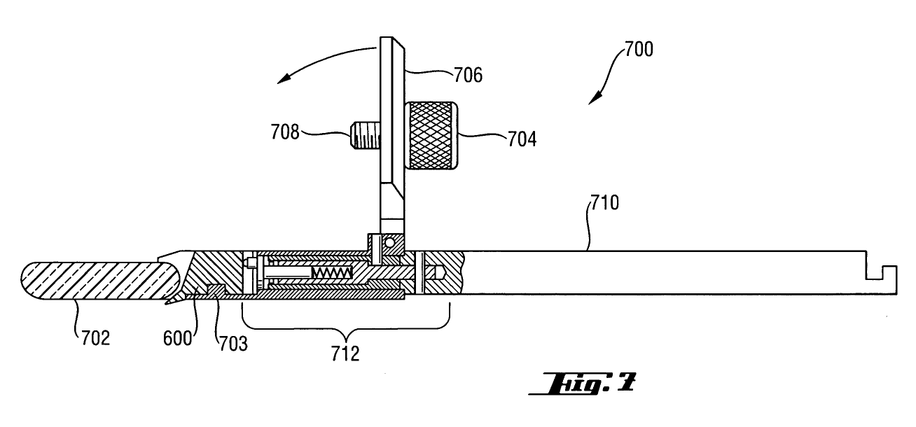

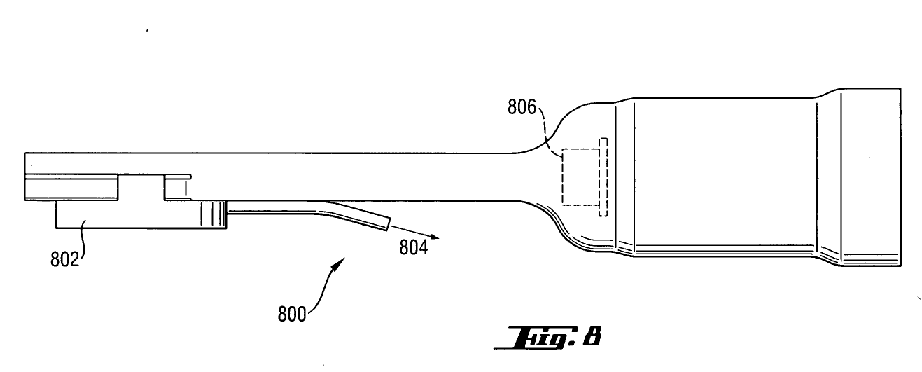

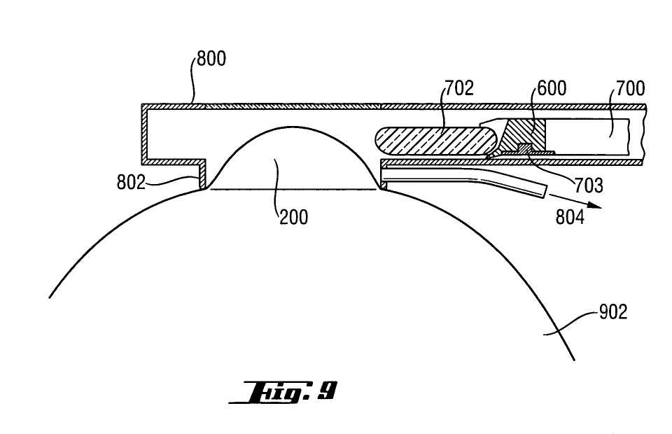

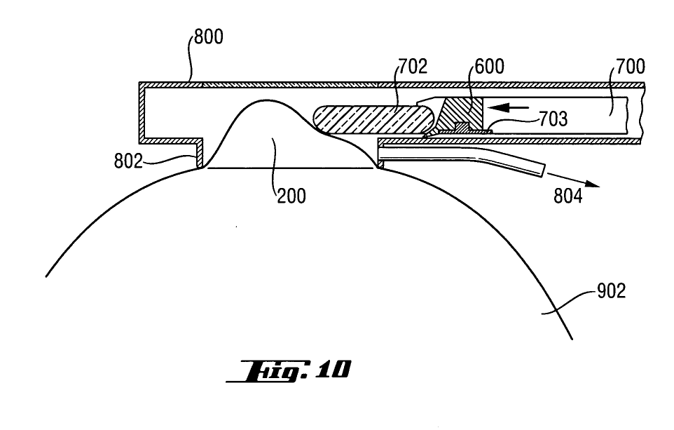

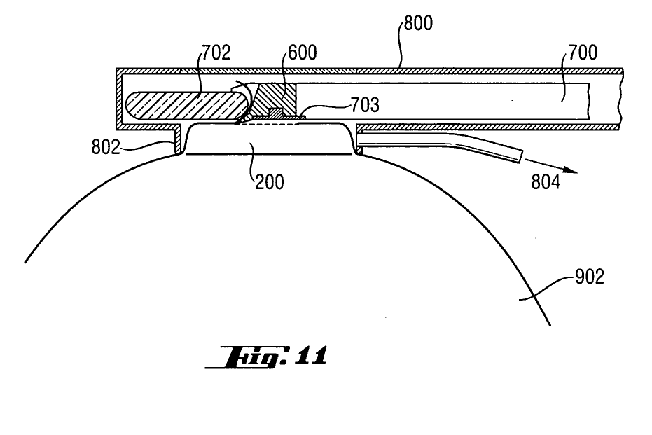

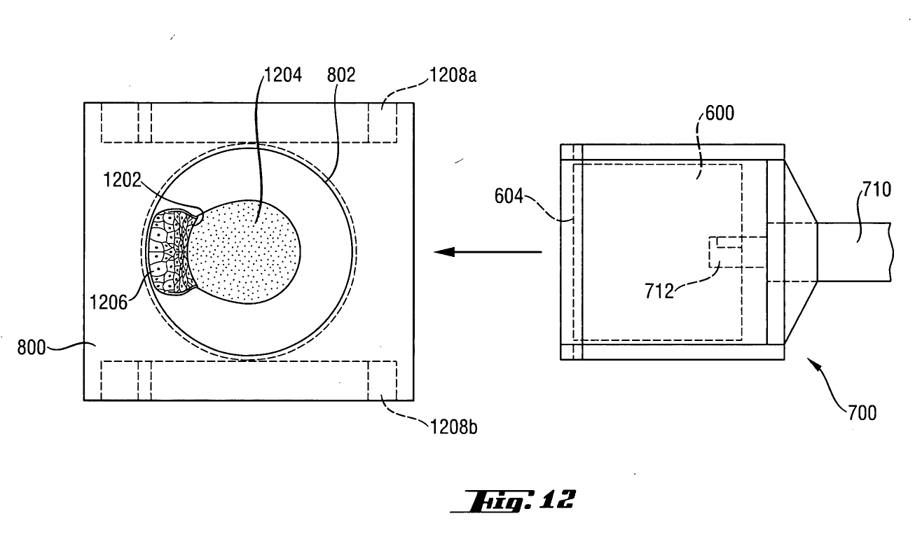

Referring to FIGS. 7-9 and 12, one embodiment of a surgical device according to the present invention comprises a hand piece 800 with an integral vacuum ring 802 and a separator assembly 700. (Note that, for simplicity, the separator cover 706 is not shown in FIGS. 9-11 and that the figures are not necessarily drawn to scale.) Separator assembly 700 comprises a drive shaft 710 that engages a motor (not shown) through a bushing 806 in the hand piece 800 to move the separator assembly 700 transversely and to oscillate the separator 600. Vacuum is applied to the vacuum ring 802 through vacuum port 804 to secure the eye thereto.

Preferably, one or more motors (not shown) provide two types of motion to the separator assembly 700 and the separator 600. The first type of motion is side-to-side oscillation along an axis parallel to the separating edge 604 of the separator 600 to assist in the separation process. The second type of motion is longitudinal motion generally perpendicular to the separating edge 604 of the separator 600 to advance the separation along the cornea. The rotational motion of the motor is transferred from the drive shaft 710 to the plunger assembly 712, through which it is translated to oscillations in the separator 600. Under action from the plunger assembly 712, the separator 600 is oscillated by the motor. The separator 600 can oscillate either transversely, vertically, or longitudinally with frequency ranging from about 10 Hz to about 10 KHz. Electromagnetic or piezoelectric forces on the separator 600 can alternatively provide the oscillation, or external rotating or vibrating wires can provide the oscillation. The separator 600 is preferably oscillated along the separator support 703 in a direction perpendicular to the plane of the figure.

An applanator 702 is connected to the separator assembly 700 in a position forward of the separator 600. Separator 600 is held firmly within the separator assembly 700 by separator cover 706, which is preferably hingedly connected to the hand piece 700 moveable in the direction of the arrow in FIG. 7. The cover 706 is secured in place through a locking screw 708, which can be tightened by hand through the locking screw head 704.

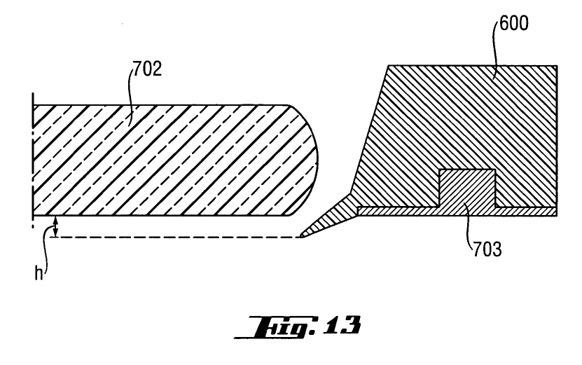

Separator assembly 700 is slidably associated with hand piece 800 through grooves 1208a, 1208b (see FIG. 12). FIG. 9 shows a cross-sectional side view of an eye 902 of a patient and an epithelial separator device comprising the hand piece 800 associated with the separator assembly 700. When the eye 902 is placed within the vacuum ring 802 and a vacuum is applied to vacuum port 804, the surface of the eye 902 is tightened and pulled through the ring 802 to expose the cornea 200 at a position forward of the applanator 702. As shown in FIG. 9, the separator assembly 700 begins in a first position located away from the eye 902.

Referring now to FIG. 10, as the applanator 702 moves forward under action of the drive shaft 700 through tracks 1208a, 1208b, the cornea 200 is forced against the undersurface of the applanator 702. This results in a flattening of the cornea 200 before it comes into contact with the separator 600. As the separator assembly 600 moves along the cornea 200 of the eye 902, the separator 600 engages the cornea 200 and removes the epithelium layer 202 located at the surface of the cornea 200 of the eye 902. However, the separator 600 is not sharp enough to excise Bowman's layer 204 during operation of the epithelial separator device, and the separator passes over the intact Bowman's layer as it separates the epithelium.

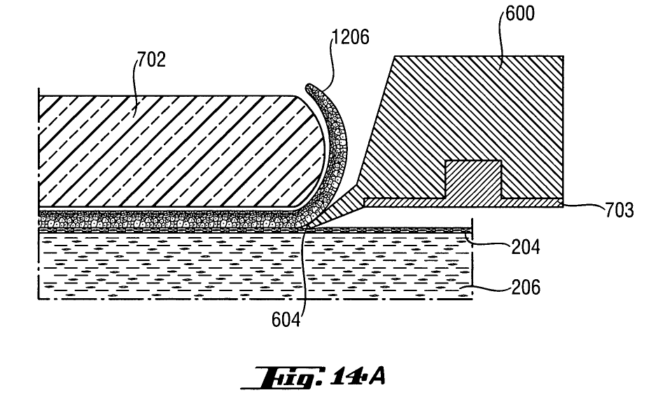

Referring now to FIG. 13, the separating edge 604 is preferably positioned or angled such that it is located at a height h below the bottom surface of the applanator 702. This spacing or distance between the separating edge and the bottom surface of the applanator does not determine the depth of the cut, as with previously known methods and devices for severing the cornea for LASIK procedures. Therefore, the exact value of this distance is not as critical to performance of the separator as it can be with previously known devices and procedures, where tens of microns can be the difference between a successful flap and a medical emergency. While prior art LASIK microkeratomes typically cut at a distance of 130-150 microns, the present separator can be set at a depth (h) from between about 40 microns to about 300 microns, more preferably from about 40 to about 100 microns. Surprisingly, consistent epithelium removal has been demonstrated at depths of about 240 microns.

The separator 600 is preferably fabricated from a synthetic polymeric material. The preferred polymeric material is a thermoplastic or thermoset polymer or ionomer. There are presently available a wide variety of durable, resilient polymers which may be employed to fabricate the separator. Included among such materials are, but are not limited to, acetals, (meth)acrylates, acrylics, alkyds, polycarbonates, polyolefins, polyesters and co-polyesters, polymethylpentene, polypropylene, polysulfones, cellulosics, styrene acrylic co-polymers, fluoropolymers, nylons, polystyrene, polyetheretherketones (PEEK), polyarylates, polyetherimides, styrene acrylonitrile, silicones, epoxys, polyvinyl chloride, urethanes, acrylonitrile-butadiene-styrene (ABS), methylmethacrylate-acrylonitrile-butadiene-styrene (MABS), allyl diglycolcarbonate, as well as combinations or blends of these polymers. The preferred polymeric materials are polycarbonates, PEEK, polystyrenes, MABS, acetal homopolymers, and poly(methyl methacrylate) (PMMA). It has in fact been found, in accord with the principles of the present invention, that many of these materials can retain a sufficiently sharp edge and have sufficient durability and resiliency to function as a separator.

Preferably, the separator has a flexural modulus of at least about 1.5 GPa according to ASTM D790-02, more preferably at least about 2.0 GPa, and most preferably at least about 3.0 GPa. Furthermore, the separator preferably has a tensile strength at yield of at least about 25 MPa according to ASTM D638-02, more preferably at least about 40 MPa, and most preferably at least about 50 MPa. Additionally, the separator preferably has either a Rockwell M hardness greater than or equal to 70 or a Rockwell R hardness greater than or equal to 90, according to ASTM D785-98e1. Most preferably, the material has a Rockwell M hardness of greater than 90. Such relatively stiff materials (compared to other plastics) are preferred in order to avoid deformation of the separator during normal operation. However, it is indeed surprising that such materials having strength and hardness less than stainless steel are nonetheless suitable for use in a separator in the present invention. Commercially available materials meeting the above preferred criteria include various grades and formulations of PEEK, PMMA, acetal homopolymer, polystyrene, MABS, and polycarbonate.

In addition to the stiffness of the material, the toughness of the material can be important in the use of the separator. Accordingly, the separator preferably has a toughness of at least about 1 J/cm², more preferably at least about 2 J/cm², most preferably at least about 3 J/cm², according to ISO 179-1 (15 Dec. 2000) Charpy Impact Test. When this test method is referenced to herein it is meant to refer only to the portion of the test performed at 23° C. using unnotched specimens. Such relatively tough materials (compared to other plastics) are preferred in order to avoid cracking or shattering of the separator during normal operation. However, it is indeed surprising that such materials having toughness less than stainless steel are nonetheless suitable for use in a separator in the present invention. Commercially available materials meeting the above preferred criteria include various grades and formulations of PEEK, PMMA, acetal homopolymer, polystyrene, MABS, and polycarbonate. However, while unmodified polystyrene has moderate strength, it is rigid and brittle. Impact strength is increased significantly by blending the polymer with rubbers such as polybutadiene. The preferred MABS is available commercially from BASF as Terlux® 2802 and the preferred polystyrene is commercially available from Nova Chemicals as Crystal PS 3500. Table 1 below presents data provided by the manufacturer of various polymers.

TABLE 1

| | Tensile | Flexural | Charpy | Vicat |
|---|---|---|---|---|
| | Strength at | Modulus | Impact | Softening |
| | Yield (MPa) | (GPa) | (J/cm²) | (° C.) |
| Terlux ® 2802 | 48 | * | 15 | 91 |
| Crystal PS 3500 | 36 | 3.5 | * | 92 |
| Victrex PEEK | 97 | 4.1 | * | * |
| 450G | | | | |
| BASF Lucryl ® KR | 60 | * | 5 | 106 |
| 2008/1 PMMA | | | | |

*Data not provided by manufacturer

In example embodiments, the polymeric material is reinforced by incorporation of various inorganic filler materials. For example, carbon and glass fibers and powders have been incorporated into various polymeric materials to greatly increase flexural strength. Such materials typically have high degrees of strength and are capable of taking and maintaining a sufficient separating edge, as well as providing sufficient toughness to allow for their use in fabricating the separating device.

In other example embodiments of the invention, the polymeric material of the separator is transparent. A transparent separator will not obstruct the visual field when observing the progress of the separator through the cornea. The polymeric material preferably exhibits a light transmission greater than 50 percent, more preferably greater than 75 percent, and a haze factor less than about 25 percent, more preferably less than about 5 percent, in accordance with ASTM D1003-00. More preferably, the polymeric material comprises a slight tint so that there it is visibly different in perceived color than the epithelium. This is easily accomplished, for example, by addition of a tinting agent to the polymer before manufacture. The slight tint will provide a contrast between the blade and the epithelium enabling the surgeon to differentiate therebetween, but yet, still providing optical clarity for observation of the cornea during use. The tint, by increasing the visibility of the separator during use, will also make it easier for the surgeon to handle the blade prior to insertion into the surgical device.

The tinting agent can include one or more pigments. Preferably, the pigment is a white pigment, a black pigment, a blue pigment, a brown pigment, a cyan pigment, a green pigment, a violet pigment, a magenta pigment, a red pigment, or a yellow pigment, or shades or combinations thereof. Suitable classes of colored pigments include, for example, anthraquinones, phthalocyanine blues, phthalocyanine greens, diazos, monoazos, pyranthrones, perylenes, heterocyclic yellows, quinacridones, diketopyrolo-pyroles, and (thio) indigoids. Representative examples of phthalocyanine blues include copper phthalocyanine blue and derivatives thereof (Pigment Blue 15). Representative examples of quinacridones include Pigment Orange 48, Pigment Orange 49, Pigment Red 122, Pigment Red 192, Pigment Red 202, Pigment Red 206, Pigment Red 207, Pigment Red 209, Pigment Violet 19 and Pigment Violet 42. Representative examples of anthraquinones include Pigment Red 43, Pigment Red 194 (Perinone Red), Pigment Red 216 (Brominated Pyanthrone Red) and Pigment Red 226 (Pyranthrone Red). Representative examples of perylenes include Pigment Red 123 (Vermillion), Pigment Red 149 (Scarlet), Pigment Red 179 (Maroon), Pigment Red 190 (Red), Pigment Violet, Pigment Red 189 (Yellow Shade Red) and Pigment Red 224. Representative examples of thioindigoids include Pigment Red 86, Pigment Red 87, Pigment Red 88, Pigment Red 181, Pigment Red 198, Pigment Violet 36, and Pigment Violet 38. Representative examples of heterocyclic yellows include Pigment Yellow 1, Pigment Yellow 3, Pigment Yellow 12, Pigment Yellow 13, Pigment Yellow 14, Pigment Yellow 17, Pigment Yellow 65, Pigment Yellow 73, Pigment Yellow 74, Pigment Yellow 110, Pigment Yellow 117, Pigment Yellow 128, Pigment Yellow 138, and Pigment Yellow 151. A representative example of diketopyrolo-pyroles include Pigment Red 254. Such pigments are commercially available in either powder or press cake form from a number of sources including, BASF Corporation, Engelhard Corporation and Sun Chemical Corporation. Examples of other suitable colored pigments are described in the Colour Index, 3rd edition (The Society of Dyers and Colourists, 1982).

In further example embodiments, the separator is constructed of a polymeric material that will undergo dimensional change if exposed to temperatures exceeding about 121° C., and more preferably if exposed to temperatures exceeding about 100° C. Such a separator is incapable of being re-used if it is autoclaved after use, because the dimensional change will render the separator incompatible for coupling with the surgical device, thereby ensuring that separators are not reused. In example embodiments, the polymeric material has a Vicat softening point, measured by ASTM D1525-00, of less than about 121° C., and more preferably of less than about 100° C. The Vicat softening point is the temperature at which a flattened needle of 1 mm² cross-section, and under a specified constant load, penetrates a specimen of the plastic to a depth of 1 mm. It is useful as a rough comparative guide to a resin's resistance to elevated temperatures.

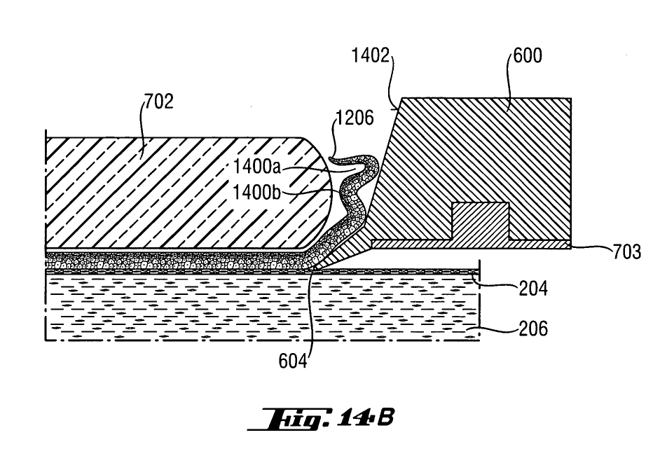

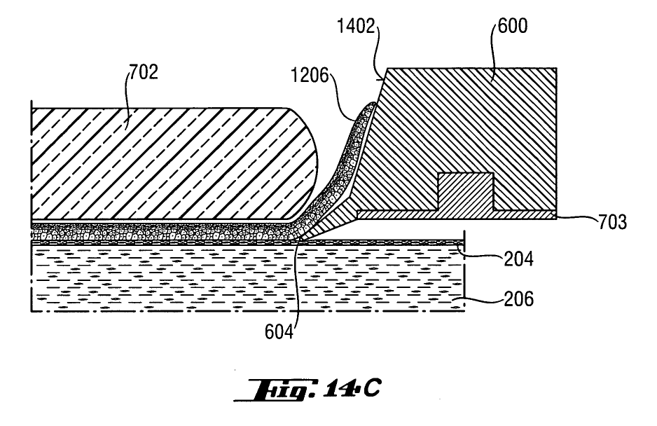

Referring to FIG. 14A, the separator 600 is used with a surgical device that separates the epithelium 1206 of a cornea from the underlying Bowman's layer 204 of an eye of a patient. As the separator 600 is positioned in contact with the eye, the separator edge 604 will cleave the fibrils connecting the epithelium 1206 to Bowman's layer 204, but will not slice into Bowman's layer 204. The separator 600 pushes the epithelial cells 1206 and preferably, does not exert a force that could disrupt the intercellular bonds, such as the desmosomes. As the separator edge 604 progresses along the eye, the epithelium 1206 is preferably left free to assume an unhindered position and configuration. Often, the epithelium 1206 will progress along the top surface of the applanator 702. Referring to FIG. 14B, depending, in part, on the angle of incidence of the separator 100 and the depth of encounter (h), the epithelium 1206 may be pushed out in front of the separator 100, forming multiple folds 1400a, 1400b as it progresses. Alternatively, the epithelium may progress up the front surface 1402 of the separator 600 as shown in FIG. 14C.

By not constraining the epithelium 1206 during separation, the epithelium 1206 encounters minimal stress and strain and will suffer less cell death. This is particularly important when the separator 600 is oscillated. If the epithelium 1206 is constrained or otherwise prevented from moving freely (such as being held against a surface post-separation), the oscillatory energy of the separator 600 will be absorbed, at least partially, by the epithelium 1206, causing cell disruption or death. However, a freely moving epithelium 1206 will not absorb as much energy from the oscillatory movement of the separator 600 and will maintain structural integrity.

Referring back to FIG. 12, when the separator assembly 700 is retracted from the cornea after separation has occurred, the separated epithelium layer 1206 is preferably left partially attached to the cornea of the eye by a hinge 1202. The hinge 1202 is preferably about 1 cm in length, but can differ significantly from this, provided enough of Bowman's layer 1204 is exposed to perform laser ablation. The separated epithelium 1206 typically will be laid out flat upon the exposed Bowman's layer 1204 after the separator assembly 700 is retracted. In this case, the epithelium is carefully moved to the side with forceps to the position shown prior to laser ablation.

While the invention has been described by reference to various embodiments, it will be understood that many changes and modifications can be made without departing from the scope of the invention. It is therefore intended that the foregoing detailed description be understood as an illustration of example embodiments of the invention, and not as a limiting definition of the invention. It is only the following claims, including all equivalents, which are intended to define the scope of this invention.

What is claimed is:

1. A separator for use with a surgical device that separates corneal epithelium from Bowman's layer, the surgical device including a positioning ring for temporary attachment to the eye, a separator assembly structured and disposed to carry said separator across the positioning ring, said separator having a polymeric separating edge that separates the corneal epithelium from the Bowman's layer as the separator moves across the positioning ring.

2. A separator as claimed in claim 1, where said separating edge is not sufficiently sharp to sever Bowman's layer.

3. A separator as claimed in claim 1, wherein said polymeric separating edge comprises a polymeric material selected from acetals, (meth)acrylates, acrylics, alkyds, polycarbonates, polyolefins, polyesters and co-polyesters, polymethylpentene, polypropylene, polysulfones, cellulosics, styrene acrylic co-polymers, fluoropolymers, nylons, polystyrene, polyetheretherketones (PEEK), polyarylates, polyetherimides, styrene acrylonitrile, silicones, epoxys, polyvinyl chloride, urethanes, acrylonitrile-butadiene-styrene (ABS), methylmethacrylate-acrylonitrile-butadiene-styrene (MABS), allyl diglycolcarbonate, and combinations thereof.

4. A separator as claimed in claim 1, wherein said polymeric separating edge comprises a polymeric material selected from polycarbonates, PEEK, polystyrenes, MABS, acetal homopolymers, PMMA, and combinations thereof.

5. A separator as claimed in claim 1, wherein said polymeric separating edge comprises a polymeric material having a flexural modulus of at least about 1.5 GPa according to ASTM D790.

6. A separator as claimed in claim 1, wherein said polymeric separating edge comprises a polymeric material having a tensile strength at yield of at least about 25 MPa according to ASTM D638.

7. A separator as claimed in claim 1, wherein said polymeric separating edge comprises a polymeric material having either a Rockwell M hardness greater than or equal to 70, or a Rockwell R hardness greater than or equal to 90, according to ASTM 785.

8. A separator as claimed in claim 1, wherein said polymeric separating edge comprises a polymeric material having a toughness of at least about 1 J/cm², according to ISO 179 Charpy Impact Test, unnotched at 23° C.

9. A separator as claimed in claim 1, wherein said polymeric separating edge comprises a polymeric material and an inorganic filler material selected from carbon powder, carbon fibers, glass powder, glass fibers, and combinations thereof.

10. A separator as claimed in claim 1, wherein said polymeric separating edge comprises a transparent material.

11. A separator as claimed in claim 10, wherein said transparent material has a light transmission greater than about 50 percent, and a haze factor less than about 25 percent, in accordance with ASTM D1003.

12. A separator as claimed in claim 10, wherein said transparent material further comprises a tinting agent.

13. A separator as claimed in claim 1, wherein said polymeric separating edge comprises a polymeric material having a Vicat softening point, measured by ASTM D1525, of less than 120° C.

14. A separator for separating corneal epithelium from Bowman's layer in a cornea of an eye, said separator comprising a polymeric separating edge that will not cut into Bowman's layer as the separator separates the corneal epithelium from the Bowman's layer.

15. A separator as claimed in claim 14, wherein said polymeric separating edge comprises an acetal, an acrylic, an alkyd, a polycarbonate, a polyesters or co-polyester, polymethylpentene, polypropylene, a polysulfone, a cellulosic, a styrene acrylic co-polymer, a fluoropolymer, nylon, polystyrene, a polyetheretherketone (PEEK), a polyarylate, a polyetherimide, styrene acrylonitrile, a silicone, epoxys, polyvinyl chloride, a urethane, acrylonitrile-butadiene-styrene (ABS), methylmethacrylate-acrylonitrile-butadiene-styrene (MABS), allyl diglycolcarbonate, or a combination thereof.

16. A separator as claimed in claim 14, wherein said polymeric separating edge comprises polycarbonate, PEEK, polystyrene, MABS, an acetal homopolymer, PMMA, or a combination thereof.

17. A separator as claimed in claim 14, wherein said polymeric separating edge comprises a material having a flexural modulus of at least about 1.5 GPa according to ASTM D790.

18. A separator as claimed in claim 14, wherein said polymeric separating edge comprises a material having a tensile strength at yield of at least about 25 MPa according to ASTM D638.

19. A separator as claimed in claim 14, wherein said polymeric separating edge comprises a material having either a Rockwell M hardness greater than or equal to 70, or a Rockwell R hardness greater than or equal to 90, according to ASTM 785.

20. A separator as claimed in claim 14, wherein said polymeric separating edge comprises a material having a toughness of at least about 1 J/cm², according to ISO 179 Charpy Impact Test, unnotched at 23° C.

21. A separator as claimed in claim 14, wherein said polymeric separating edge further comprises an inorganic filler material selected from the group of carbon powder, carbon fibers, glass powder, glass fibers, and combinations thereof.

22. A separator as claimed in claim 14, wherein said polymeric separating edge is transparent.

23. A separator as claimed in claim 22, wherein said polymeric separating edge comprises a material having a light transmission greater than about 50 percent, and a haze factor less than about 25 percent, measured in accordance with ASTM D1003.

24. A separator as claimed in claim 23, wherein said material further comprises a tinting agent.

25. A separator as claimed in claim 14, wherein said polymeric separating edge comprises a material having a Vicat softening point, as measured by ASTM D1525, of less than 120° C.

26. A separator for separating a corneal epithelium from an eye, at least a leading edge portion of the separator comprising a polymeric material, the leading edge portion of the separator having an edge thickness of at least about 200 microns.

27. The separator of claim 26, further comprising at least one surface feature for coupling with a surgical device.

28. A disposable separator for separating a corneal epithelium from an eye, the separator having a leading edge with a radius of curvature of between about 5 microns and about 100 microns.

29. The disposable separator of claim 28, wherein the radius of curvature of the leading edge of the separator is between about 10 microns and about 30 microns.

30. The disposable separator of claim 28, wherein the radius of curvature of the leading edge of the separator is between about 15 microns and about 25 microns.

31. A method for separating at least a portion of an epithelium from a cornea of an eye, so that an intact Bowman's layer is exposed, said method comprising the steps of:
(a) fixing a positioning ring to an eye so that the cornea at least partially extends therethrough;
(b) moving a separator having a polymeric separating edge across at least a portion of the cornea to separate the epithelium from the cornea, leaving Bowman's layer intact; and
(c) retracting the separator outside the positioning ring.

32. A method as claimed in claim 31, further comprising the step of flattening at least a portion of the cornea prior to moving the separator along the travel path.

33. A method of preventing re-use of a surgical device for tissue separation, said method comprising forming at least a working edge portion of said surgical device of a polymeric material that will deform at a temperature of less than about 121° C.

34. The method of claim 33, wherein the polymeric material of the working edge portion deforms at a temperature of less than about 100° C.
